(19) United States
(12) Patent Application Publication
(10) Pub. No.: US 2015/0223463 A1
(43) Pub. Date: Aug. 13, 2015

(54) SPIROCYCLIC ISOXAZOLINE DERIVATIVES FOR TREATMENT OF SEA LICE

(71) Applicant: Zoetis LLC, Florham Park, NJ (US)

(72) Inventors: John Adam Wendt, Kalamazoo, MI (US); David Jose Asper, Kalamazoo, MI (US)

(21) Appl. No.: 14/423,288

(22) PCT Filed: Sep. 3, 2013
(86) PCT No.: PCT/US2013/057787

§ 371 Date: Feb. 23, 2015

Related U.S. Application Data
(60) Provisional application No. 61/696,423, filed on Sep. 4, 2012.

(51) Int. Cl. A01N 43/90; A61K 39/00; A23K 1/16; A01N 25/00; A23K 1/18; A61K 31/422; A61K 45/06

(52) U.S. Cl. CPC A01N 43/90; A61K 31/422; A61K 39/00; A61K 45/06; A01N 25/002; A23K 1/188; A23K 1/1628; A61K 2039/521; A61K 2039/5252

(57) ABSTRACT

The invention recites a method of treating a parasitic infection in fish comprising administering an effective amount of a spirocyclic isoxazoline compound of Formula 1, stereoisomers thereof, and veterinary acceptable salts thereof, for use against sea lice in a fish, and compositions thereof, (formula I), wherein W, X, R^{1a}, R^{1b}, R^{1c}, R², R³, R⁴, n, and “*” are as defined herein.

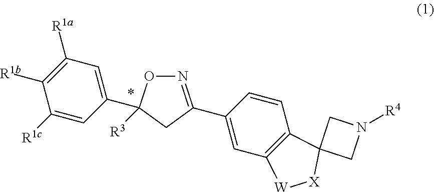

### FIELD OF THE INVENTION

This invention relates to spirocyclic isoxazoline derivatives having activity against sea lice in fish.

### BACKGROUND

Sea lice are parasitic crustaceans/copepods within the order Siphonostomatoida, family Caligidae that feed on the mucus, epidermal tissue, and blood of host marine fish. Johnson et al, Parasitol Res (2002) 88: 789-796. Sea lice are prevalent parasites, particularly on salmonids, and, when present in high numbers, can cause serious disease and ultimately host death. In fish farms, where highly concentrated fish populations are present, sea lice can have a devastating effect on the stock.

In 2006, total salmonid marine production was 1.7 million tons, worth US $8.4 billion. See FAO Fisheries and Aquaculture Information and Statistics Service 2008, Aquaculture Production 1950-2006. Available data indicates sea lice cost from [symbol] 0.1 to [symbol] 0.2 kg⁻¹ of fish. Mark J Costello, The global economic cost of sea lice to the salmonid farming industry, Journal of Fish Diseases, v. 32(1), pgs 115-118 (2009). However, without treatment measures, sea lice would cost the industry at least four times more and probably increase to levels such as to cause significant direct and indirect mortality to stock. Mustafa et al, Canadian Veterinary Journal 42, 54-56 (2001). Existing regional estimates for the cost of sea lice ranged from 4% of production value for Atlantic Canada to 7-10% in Scotland. Rae et al, Pest Management Science 58, 515-520 (2002)). Notably, Costello et al., supra, indicates a cost of 6% of the value of fish production for the countries affected by sea lice.

To date, available treatment regimens against sea lice infestations have been very limited with macrocyclic lactones, such as emamectin benzoate (SLICE®), being the only significant commercial treatment available. Additional ectoparasiticidal compounds have been explored, such as those described in U.S. Publication No. 2010-0303865, but no successful treatment agents based on this disclosure have emerged. Additionally, studies have been conducted on vaccine compositions targeting antigens present in sea lice, but no products using the vaccine approach have emerged either.

Accordingly, SLICE® has been widely used and as a result, significant resistance amongst sea lice populations has arisen. Additionally, macrocyclic lactones are observed to have high toxicity and deleterious environmental effects due to their non-selective activity against other benign microorganisms.

Accordingly, a need exists for a novel agent capable of treating sea lice infestations in fish, particularly in farmed salmon populations, that is safe and selective against the target parasite, without causing collateral ecological damage to other marine organisms.

Isoxazoline derivatives have been disclosed in the art as having insecticidal and acaricidal activity. For example, WO2007/105814, WO2008/122375, and WO2009/035004 recite certain alkylene linked amides. Further, WO2007/075459, WO2010/084067 and WO2010/025998, disclose phenyl isoxazolines substituted with a 5- to 6-membered heterocycle, and/or 10- to 11-membered fused aryl and heteroaryl substitutions. However, none of these citations exemplify spirocyclic substituted isoxazolines, or processes of manufacturing the spirocyclic compounds. WO2012/120399 describes similar compounds of the instant invention, including processes for making them, however, the citation does not describe their use in aquaculture. The process for preparing the polymorphic Form A of (S)-1-(5-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone is described in PCT/IB2012/050842.

The present invention overcomes one or more of the various disadvantages of, or improves upon, the properties of existing compounds. In particular the present invention develops new spirocyclic isoxazoline substituted azetidine compounds which demonstrate potent activity against sea lice.

### SUMMARY

The present invention provides a method for the treatment of a parasitic infection or infestation in a fish comprising administering to said fish an effective amount of a compound of Formula 1

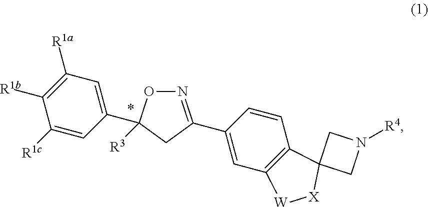

wherein

X and W are each independently —O—, —S—, —NR⁶—, —CH₂—, —C(O)—, —C(NR⁷)—, or —C(S)—, when X is —O—, —S—, or —NR⁶—, then W is —CH₂—, —C(O)—, —C(NR⁷)—, or —C(S)—, and when W is —O—, —S—, or —NR⁶—, then X is —CH₂—, —C(O)—, —C(NR⁷)—, or —C(S)—;

R^{1a}, R^{1b}, and R^{1c} are each independently hydrogen, halo, hydroxyl, cyano, nitro, C₁-C₆alkyl, C₁-C₆haloalkyl, C₁-C₆alkoxy, C₀-C₃alkylC₃-C₆ cycloalkyl, C₁-C₆haloalkoxy, —C(O)NH₂, —SF₅, or —S(O)ₚR;

R² is fluoro, chloro, or C₁-C₆alkyl;

R³ is cyano, C₁-C₆alkyl, C₁-C₆haloalkyl, —C(O)NR^{a}R^{b}, C₂-C₆alkenyl, C₂-C₆alkynyl, C₂-C₆haloalkenyl, or C₂-C₆haloalkynyl

R⁴ is hydrogen, C₁-C₆alkyl, C₀-C₆alkylC₃-C₆cycloalkyl, —C(O)R⁵, —C(S)R⁵, —C(O)NR^{a}R⁵, —C(O)C(O)NR^{a}R⁵, —S(O)ₚR^{c}, —S(O)₂NR^{a}R⁵, —C(NR⁷)R⁵, —C(NR⁷)NR^{a}R⁵, C₀-C₆alkylphenyl, C₀-C₆alkylheteroaryl, or C₀-C₆alkylheterocycle;

R⁵ is hydrogen, C₁-C₆alkyl, C₂-C₆alkenyl, C₀-C₆alkylC₃-C₆cycloalkyl, C₀-C₆alkylphenyl, C₀-C₆alkylheteroaryl, or C₀-C₆alkylheterocycle;

R⁶ is hydrogen, C₁-C₆alkyl, hydroxyl, or C₁-C₆alkoxy;

R⁷ is hydrogen, C₁-C₆alkyl, hydroxyl, cyano, nitro, —S(O)ₚR^{c}, or C₁-C₆alkoxy;

R is C₁-C₆alkyl or C₃-C₆cycloalkyl optionally substituted with at least one halo substituent;

R^{a} is hydrogen, C₁-C₆alkyl, or C₀-C₃alkylC₃-C₆cycloalkyl; wherein the alkyl and alkylcycloalkyl is optionally substituted by cyano or at least one halo substituent;

R^{b} is hydrogen, C₁-C₆alkyl, C₃-C₆cycloalkyl, C₀-C₃alkylphenyl, C₀-C₃alkylheteroaryl, or C₀-C₃alkylheterocycle, each optionally substituted, where chemically possible, with at least one substituent selected from hydroxyl, cyano, halo, or —S(O)ₚR;

R^{c} is C₁-C₆alkyl, C₁-C₆haloalkyl, C₁-C₆haloalkylC₃-C₆cycloalkyl, C₀-C₃alkylC₃-C₆cycloalkyl, C₀-C₃alkylphenyl, C₀-C₃alkylheteroaryl, or C₀-C₃alkylheterocycle each optionally substituted with at least one substituent selected from cyano, halo, hydroxyl, oxo, C₁-C₆alkoxy, C₁-C₆haloalkoxy, C₁-C₆haloalkyl, —S(O)ₚR, —SH, —S(O)ₚNR^{a}R^{b}, —NR^{a}R^{b}, —NR^{a}C(O)R^{b}, —SC(O)R, —SCN, or —C(O)NR^{a}R^{b};

each of R⁴ and R⁵ C₁-C₆alkyl or C₀-C₆alkylC₃-C₆cycloalkyl moiety can be optionally and independently substituted by at least one substituent selected from cyano, halo, hydroxyl, oxo, C₁-C₆alkoxy, C₁-C₆haloalkoxy, C₁-C₆haloalkyl, C₁-C₆alkyl, hydroxylC₁-C₆alkyl-, —S(O)ₚR^{c}, —SH, —S(O)ₚNR^{a}R^{b}, —NR^{a}R^{b}, —NR^{a}C(O)R^{b}, —SC(O)R, —SCN, or —C(O)NR^{a}R^{b}; and

wherein each of R⁴ and R⁵ is C₀-C₆alkylphenyl, C₀-C₆alkylheteroaryl, or C₀-C₆alkylheterocycle moiety can be further optionally substituted with at least one substituent selected from cyano, halo, oxo, ═S, =NR⁷, hydroxylC₁-C₆alkyl-, hydroxyl, C₁-C₆alkoxy, C₁-C₆alkyl, C₁-C₆haloalkyl, —SH, —S(O)ₚR, and C₁-C₆haloalkoxy;

n is the integer 0, 1, or 2, and when n is 2, each R² may be identical or different from each other;

p is the integer 0, 1, or 2; and

wherein “*” is a chiral center, stereoisomers thereof, and veterinarily acceptable salts thereof.

In another aspect of the invention, is a method for the treatment of a parasitic infection or infestation in a fish comprising administering to said fish an effective amount of a compound of Formula 1, selected from:
1-(cyclopropanecarbonyl)-5′-(3,4,5-trichlorophenyl)-5-(trifluoromethyl)-4,5-dihydroisoxazol-3-yl)-3′H-spiro{azetidine-3,1′-isobenzofuran}-3′-one;
5′-5-(3,5-dichloro-4-fluorophenyl)-5-(trifluoromethyl)-4,5-dihydroisoxazol-3-yl)-1-propionyl-3′H-spiro[azetidine-3,1′-isobenzofuran]-3′-one;
1-(cyclopropanecarbonyl)-5′-5-(3,5-dichloro-4-fluorophenyl)-5-(trifluoromethyl)-4,5-dihydroisoxazol-3-yl)-3′H-spiro[azetidine-3,1′-isobenzofuran]-3′-one;
5′-(5-(3,5-dichloro-4-fluorophenyl)-5-(trifluoromethyl)-4,5-dihydroisoxazol-3-yl)-1-(3-methylbutanoyl)-3′H-spiro[azetidine-3,1′-isobenzofuran]-3′-one;
5′-(5-(3,5-dichloro-4-fluorophenyl)-5-(trifluoromethyl)-4,5-dihydroisoxazol-3-yl)-1-(2-hydroxy-2-methylpropanoyl)-3′H-spiro[azetidine-3,1′-isobenzofuran]-3′-one;
1-(2-cyclopropylacetyl)-5′-5-(3,5-dichloro-4-fluorophenyl)-5-(trifluoromethyl)-4,5-dihydroisoxazol-3-yl)-3′H-spiro[azetidine-3,1′-isobenzofuran]-3′-one;
1-acetyl-5′-5-(3,5-dichloro-4-fluorophenyl)-5-(trifluoromethyl)-4,5-dihydroisoxazol-3-yl)-3′H-spiro[azetidine-3,1′-isobenzofuran]-3′-one;
5′-5-(3,5-dichloro-4-fluorophenyl)-5-(trifluoromethyl)-4,5-dihydroisoxazol-3-yl)-1-(1-hydroxycyclopropanecarbonyl)-3′H-spiro[azetidine-3,1′-isobenzofuran]-3′-one;
1-(cyclobutanecarbonyl)-5′-5-(3,5-dichloro-4-fluorophenyl)-5-(trifluoromethyl)-4,5-dihydroisoxazol-3-yl)-3′H-spiro[azetidine-3,1′-isobenzofuran]-3′-one;
5′-(5-(3,5-dichloro-4-fluorophenyl)-5-(trifluoromethyl)-4,5-dihydroisoxazol-3-yl)-1-pivaloyl-3′H-spiro[azetidine-3,1′-isobenzofuran]-3′-one;
5′-(5-(3,5-dichloro-4-fluorophenyl)-5-(trifluoromethyl)-4,5-dihydroisoxazol-3-yl)-1-(2-hydroxyacetyl)-3′H-spiro[azetidine-3,1′-isobenzofuran]-3′-one;
5′-5-(3,5-dichloro-4-fluorophenyl)-5-(trifluoromethyl)-4,5-dihydroisoxazol-3-yl)-1-(2-(1-hydroxycyclopropyl)acetyl)-3′H-spiro[azetidine-3,1′-isobenzofuran]-3′-one;
1-butyryl-5′-5-(3,5-dichloro-4-fluorophenyl)-5-(trifluoromethyl)-4,5-dihydroisoxazol-3-yl)-3′H-spiro[azetidine-3,1′-isobenzofuran]-3′-one;
5′-(5-(3,5-dichloro-4-fluorophenyl)-5-(trifluoromethyl)-4,5-dihydroisoxazol-3-yl)-1-(2-(methylthio)acetyl)-3′H-spiro[azetidine-3,1′-isobenzofuran]-3′-one;
5′-(5-(3,5-dichloro-4-fluorophenyl)-5-(trifluoromethyl)-4,5-dihydroisoxazol-3-yl)-1-(2-(methylsulfinyl)acetyl)-3′H-spiro[azetidine-3,1′-isobenzofuran]-3′-one;
5′-(5-(3,5-dichloro-4-fluorophenyl)-5-(trifluoromethyl)-4,5-dihydroisoxazol-3-yl)-1-(2-(methylsulfonyl)acetyl)-3′H-spiro[azetidine-3,1′-isobenzofuran]-3′-one;
1-(2-(1H-pyrazol-1-yl)acetyl)-5′-(5-(3,5-dichloro-4-fluorophenyl)-5-(trifluoromethyl)-4,5-dihydroisoxazol-3-yl)-3′H-spiro[azetidine-3,1′-isobenzofuran]-3′-one;
5′-5-(3,5-dichloro-4-fluorophenyl)-5-(trifluoromethyl)-4,5-dihydroisoxazol-3-yl)-1-(1-(trifluoromethyl)cyclopropanecarbonyl)-3′H-spiro[azetidine-3,1′-isobenzofuran]-3′-one;
5′-(5-(3,5-dichloro-4-fluorophenyl)-5-(trifluoromethyl)-4,5-dihydroisoxazol-3-yl)-1-isobutyryl-3′H-spiro[azetidine-3,1-isobenzofuran]-3′-one;
5′-5-(3,5-dichloro-4-fluorophenyl)-5-(trifluoromethyl)-4,5-dihydroisoxazol-3-yl)-1-(2-(3-methyl-1H-pyrazol-1-yl)acetyl)-3′H-spiro[azetidine-3,1′-isobenzofuran]-3′-one;
5′-(5-(3,5-dichloro-4-fluorophenyl)-5-(trifluoromethyl)-4,5-dihydroisoxazol-3-yl)-1-(3-hydroxy-2-methylpropanoyl)-3′H-spiro[azetidine-3,1′-isobenzofuran]-3′-one;
5′-5-(3,5-dichloro-4-fluorophenyl)-5-(trifluoromethyl)-4,5-dihydroisoxazol-3-yl)-1-(2,2-difluorocyclopropanecarbonyl)-3′H-spiro[azetidine-3,1′-isobenzofuran]-3′-one;
5′-(5-(3,5-dichloro-4-fluorophenyl)-5-(trifluoromethyl)-4,5-dihydroisoxazol-3-yl)-1-(4,4,4-trifluorobutanoyl)-3′H-spiro[azetidine-3,1′-isobenzofuran]-3′-one;
1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-((trifluoromethyl)thio)ethanone;
(5′-5-(3,5-dichloro-4-fluorophenyl)-5-(trifluoromethyl)-4,5-dihydroisoxazol-3-yl)-3′H-spiro[azetidine-3,1′-isobenzofuran]-1-yl)(1-oxidothietan-3-yl)methanone;
(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)(1,1-dioxidothietan-3-yl)methanone;
1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylthio)ethanone;
(R)-1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylthio)ethanone;
(S)-1-(5′-5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylthio)ethanone;
1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfinyl)ethanone;
(R)-1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfinyl)ethanone;
(S)-1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfinyl)ethanone;
1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone;
(R)-1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone;
(S)-1-(5′-5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone;
1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-methyl propan-1-one;
1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-hydroxyethanone;
cyclobutyl(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)methanone;
(5′-5-(3,5-dichloro-4-fluorophenyl)-5-(trifluoromethyl)-4,5-dihydroisoxazol-3-yl)-3′H-spiro[azetidine-3,1′-isobenzofuran]-1-yl)(1-hydroxycyclopropyl)methanone;
N-(2-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-oxoethyl)formamide;
1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)ethanone;
1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)propan-1-one;
1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-hydroxy-2-methyl propan-1-one;
2-cyclopropyl-1-(5′-5-(3,5-dichloro-4-fluorophenyl)-5-(trifluoromethyl)-4,5-dihydroisoxazol-3-yl)-3′H-spiro[azetidine-3,1′-isobenzofuran]-1-yl)ethanone;
1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2,2-dimethylpropan-1-one;
(5′-5-(3,5-dichloro-4-fluorophenyl)-5-(trifluoromethyl)-4,5-dihydroisoxazol-3-yl)-3′H-spiro[azetidine-3,1′-isobenzofuran]-1-yl)(1-(trifluoromethyl)cyclopropyl)-methanone;
1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-3-hydroxy-2-methyl propan-1-one;
1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(3-methyl-1H-pyrazol-1-yl)ethanone;
1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-3-methylbutan-1-one;
1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(1H-pyrazol-1-yl)ethanone;
1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-3-hydroxybutan-1-one;
cyclopropyl(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)methanone;
1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)butan-1-one;
(5′-5-(3,5-dichloro-4-fluorophenyl)-5-(trifluoromethyl)-4,5-dihydroisoxazol-3-yl)-3′H-spiro[azetidine-3,1′-isobenzofuran]-1-yl)(thietan-3-yl)methanone;
(5′-(5-(3,5-dichloro-4-fluorophenyl)-5-(trifluoromethyl)-4,5-dihydroisoxazol-3-yl)-2′,3′-dihydrospiro[azetidine-3,1′-inden]-1-yl)(1,1-dioxidothietan-3-yl)methanone;
(R)-(5′-(5-(3,5-dichloro-4-fluorophenyl)-5-(trifluoromethyl)-4,5-dihydroisoxazol-3-yl)-2′,3′-dihydrospiro[azetidine-3,1′-inden]-1-yl)(1,1-dioxidothietan-3-yl)methanone;
(S)-(5′-(5-(3,5-dichloro-4-fluorophenyl)-5-(trifluoromethyl)-4,5-dihydroisoxazol-3-yl)-2′,3′-dihydrospiro[azetidine-3,1′-inden]-1-yl)(1,1-dioxidothietan-3-yl)methanone;
2-(methylsulfonyl)-1-(5′-5-(3,4,5-trichlorophenyl)-5-(trifluoromethyl)-4,5-dihydroisoxazol-3-yl)-3′H-spiro[azetidine-3,1′-isobenzofuran]-1-yl)ethanone;
1-(5′-5-(3,5-dichlorophenyl)-5-(trifluoromethyl)-4,5-dihydroisoxazol-3-yl)-3′H-spiro[azetidine-3,1′-isobenzofuran]-1-yl)-2-(methylsulfonyl)ethanone;
1-(5′-5-(3-chloro-5-(trifluoromethyl)phenyl)-5-(trifluoromethyl)-4,5-dihydroisoxazol-3-yl)-3′H-spiro[azetidine-3,1′-isobenzofuran]-1-yl)-2-(methylsulfonyl)ethanone;
1-(5′-(5-(3,4-dichloro-5-(trifluoromethyl)phenyl)-5-(trifluoromethyl)-4,5-dihydroisoxazol-3-yl)-3′H-spiro[azetidine-3,1′-isobenzofuran]-1-yl)-2-(methylsulfonyl)-ethanone;
1-(5′-(5-(4-bromo-3,5-dichlorophenyl)-5-(trifluoromethyl)-4,5-dihydroisoxazol-3-yl)-3′H-spiro[azetidine-3,1′-isobenzofuran]-1-yl)-2-(methylsulfonyl)ethanone;
1-(5′-(5-(3,5-bis(trifluoromethyl)phenyl)-5-(trifluoromethyl)-4,5-dihydroisoxazol-3-yl)-3′H-spiro[azetidine-3,1′-isobenzofuran]-1-yl)-2-(methylsulfonyl)ethanone;
(R)-1-(5′-(5-(3,5-bis(trifluoromethyl)phenyl)-5-(trifluoromethyl)-4,5-dihydroisoxazol-3-yl)-3′H-spiro[azetidine-3,1′-isobenzofuran]-1-yl)-2-(methylsulfonyl)ethanone;
(S)-1-(5′-(5-(3,5-bis(trifluoromethyl)phenyl)-5-(trifluoromethyl)-4,5-dihydroisoxazol-3-yl)-3′H-spiro[azetidine-3,1′-isobenzofuran]-1-yl)-2-(methylsulfonyl)ethanone;
1-(5′-(5-(3-bromo-5-chlorophenyl)-5-(trifluoromethyl)-4,5-dihydroisoxazol-3-yl)-3′H-spiro[azetidine-3,1′-isobenzofuran]-1-yl)-2-(methylsulfonyl)ethanone;
1-(5′-(5-(4-chloro-3,5-bis(trifluoromethyl)phenyl)-5-(trifluoromethyl)-4,5-dihydroisoxazol-3-yl)-3′H-spiro[azetidine-3,1′-isobenzofuran]-1-yl)-2-(methylsulfonyl)ethanone;
1-(5′-(5-(3-chloro-5-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone;
1-(5′-(5-(3-chloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone;
(R)-1-(5′-(5-(3-chloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone;
(S)-1-(5′-(5-(3-chloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone;
2-(methylsulfonyl)-1-(5′-(5-(trifluoromethyl)-5-(3-(trifluoromethyl)phenyl)-4,5-dihydroisoxazol-3-yl)-3′H-spiro[azetidine-3,1′-isobenzofuran]-1-yl)ethanone;
(R)-2-(methylsulfonyl)-1-(5′-(5-(trifluoromethyl)-5-(3-(trifluoromethyl)phenyl)-4,5-dihydroisoxazol-3-yl)-3′H-spiro[azetidine-3,1′-isobenzofuran]-1-yl)ethanone;
(S)-2-(methylsulfonyl)-1-(5′-(5-(trifluoromethyl)-5-(3-(trifluoromethyl)phenyl)-4,5-dihydroisoxazol-3-yl)-3′H-spiro[azetidine-3,1′-isobenzofuran]-1-yl)ethanone;
5′-(5-(3,5-bis(trifluoromethyl)phenyl)-5-(trifluoromethyl)-4,5-dihydroisoxazol-3-yl)-1-(2-(methylsulfonyl)acetyl)spiro[azetidine-3,1′-isoindolin]-3′-one;
5′-5-(3,5-dichloro-4-fluorophenyl)-5-(trifluoromethyl)-4,5-dihydroisoxazol-3-yl)-1-(2-(methylsulfonyl)acetyl)spiro[azetidine-3,1′-isoindolin]-3′-one;
1-(cyclopropanecarbonyl)-5′-5-(3,5-dichloro-4-fluorophenyl)-5-(trifluoromethyl)-4,5-dihydroisoxazol-3-yl)spiro[azetidine-3,1-isoindolin]-3′-one;
5′-5-(3,4-dichloro-5-(trifluoromethyl)phenyl)-5-(trifluoromethyl)-4,5-dihydroisoxazol-3-yl)-1-(2-(methylsulfonyl)acetyl)spiro[azetidine-3,1′-isoindolin]-3′-one;
1-(2-(methylsulfonyl)acetyl)-5′-(5-(3,4,5-trichlorophenyl)-5-(trifluoromethyl)-4,5-dihydroisoxazol-3-yl)spiro[azetidine-3,1′-isoindolin]-3′-one;
5′-5-(3-chloro-5-(trifluoromethyl)phenyl)-5-(trifluoromethyl)-4,5-dihydroisoxazol-3-yl)-1-(2-(methylsulfonyl)acetyl)spiro[azetidine-3,1′-isoindolin]-3′-one;
5′-5-(4-bromo-3,5-dichlorophenyl)-5-(trifluoromethyl)-4,5-dihydroisoxazol-3-yl)-1-(2-(methylsulfonyl)acetyl)spiro[azetidine-3,1′-isoindolin]-3′-one;
5′-5-(3,5-dichlorophenyl)-5-(trifluoromethyl)-4,5-dihydroisoxazol-3-yl)-1-(2-(methylsulfonyl)acetyl)spiro[azetidine-3,1′-isoindolin]-3′-one;
5′-5-(3,5-dichloro-4-fluorophenyl)-5-(trifluoromethyl)-4,5-dihydroisoxazol-3-yl)-1-(thietane-3-carbonyl)spiro[azetidine-3,1′-isoindolin]-3′-one;
5′-5-(3,5-dichloro-4-fluorophenyl)-5-(trifluoromethyl)-4,5-dihydroisoxazol-3-yl)-1-(1,1-dioxidothietane-3-carbonyl)spiro[azetidine-3,1′-isoindolin]-3′-one;
5′-5-(3,5-bis(trifluoromethyl)phenyl)-5-(trifluoromethyl)-4,5-dihydroisoxazol-3-yl)-1-(cyclopropanecarbonyl)spiro[azetidine-3,1′-isoindolin]-3′-one;
5′-5-(3-chloro-5-(trifluoromethyl)phenyl)-5-(trifluoromethyl)-4,5-dihydroisoxazol-3-yl)-1-(cyclopropanecarbonyl)spiro[azetidine-3,1′-isoindolin]-3′-one;
5′-5-(4-bromo-3,5-dichlorophenyl)-5-(trifluoromethyl)-4,5-dihydroisoxazol-3-yl)-1-(cyclopropanecarbonyl)spiro[azetidine-3,1′-isoindolin]-3′-one;
2′-methyl-1-(2-(methylsulfonyl)acetyl)-5′-(5-(3,4,5-trichlorophenyl)-5-(trifluoromethyl)-4,5-dihydroisoxazol-3-yl)spiro[azetidine-3,1′-isoindolin]-3′-one;
5′-(5-(3,5-dichloro-4-fluorophenyl)-5-(trifluoromethyl)-4,5-dihydroisoxazol-3-yl)-2′-methyl-1-(2-(methylsulfonyl)acetyl)spiro[azetidine-3,1′-isoindolin]-3′-one; and
1-(cyclopropanecarbonyl)-2′-methyl-5′-5-(3,4,5-trichlorophenyl)-5-(trifluoromethyl)-4,5-dihydroisoxazol-3-yl)spiro[azetidine-3,1′-isoindolin]-3′-one, or a stereoisomer thereof, or a veterinarily acceptable salt thereof.

In another aspect of the invention, is method for the treatment of a parasitic infection or infestation in a fish comprising administering to said fish an effective amount of a compound of Formula 1 selected from:
1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylthio)ethanone;
(R)-1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylthio)ethanone;
(S)-1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylthio)ethanone;
1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfinyl)ethanone;
(R)-1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfinyl)ethanone;
(S)-1-(5′-5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfinyl)ethanone;
1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone;
(R)-1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone;
(S)-1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone;
(5′-(5-(3,5-dichloro-4-fluorophenyl)-5-(trifluoromethyl)-4,5-dihydroisoxazol-3-yl)-2′,3′-dihydrospiro[azetidine-3,1′-inden]-1-yl)(1,1-dioxidothietan-3-yl)methanone;
(R)-(5′-5-(3,5-dichloro-4-fluorophenyl)-5-(trifluoromethyl)-4,5-dihydroisoxazol-3-yl)-2′,3′-dihydrospiro[azetidine-3,1′-inden]-1-yl)(1,1-dioxidothietan-3-yl)methanone;
(S)-(5′-5-(3,5-dichloro-4-fluorophenyl)-5-(trifluoromethyl)-4,5-dihydroisoxazol-3-yl)-2′,3′-dihydrospiro[azetidine-3,1′-inden]-1-yl)(1,1-dioxidothietan-3-yl)methanone;
1-(5′-(5-(3,5-bis(trifluoromethyl)phenyl)-5-(trifluoromethyl)-4,5-dihydroisoxazol-3-yl)-3′H-spiro[azetidine-3,1′-isobenzofuran]-1-yl)-2-(methylsulfonyl)ethanone;
(R)-1-(5′-(5-(3,5-bis(trifluoromethyl)phenyl)-5-(trifluoromethyl)-4,5-dihydroisoxazol-3-yl)-3′H-spiro[azetidine-3,1′-isobenzofuran]-1-yl)-2-(methylsulfonyl)ethanone;
(S)-1-(5-(5-(3,5-bis(trifluoromethyl)phenyl)-5-(trifluoromethyl)-4,5-dihydroisoxazol-3-yl)-3′H-spiro[azetidine-3,1′-isobenzofuran]-1-yl)-2-(methylsulfonyl)ethanone;
1-(5′-(5-(3-chloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone;
(R)-1-(5′-(5-(3-chloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone;
(S)-1-(5′-(5-(3-chloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone;
2-(methylsulfonyl)-1-(5′-5-(trifluoromethyl)-5-(3-(trifluoromethyl)phenyl)-4,5-dihydroisoxazol-3-yl)-3′H-spiro[azetidine-3,1′-isobenzofuran]-1-yl)ethanone;
(R)-2-(methylsulfonyl)-1-(5-(5-(trifluoromethyl)-5-(3-(trifluoromethyl)phenyl)-4,5-dihydroisoxazol-3-yl)-3′H-spiro[azetidine-3,1′-isobenzofuran]-1-yl)ethanone; and
(S)-2-(methylsulfonyl)-1-(5′-(5-(trifluoromethyl)-5-(3-(trifluoromethyl)phenyl)-4,5-dihydroisoxazol-3-yl)-3′H-spiro[azetidine-3,1′-isobenzofuran]-1-yl)ethanone,
   or a stereoisomer thereof, or a veterinarily acceptable salt thereof.

In another aspect of the invention, is a method for the treatment of a parasitic infection or infestation in a fish comprising administering to said fish an effective amount of a compound of Formula 1 that is 1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone, having the structure:

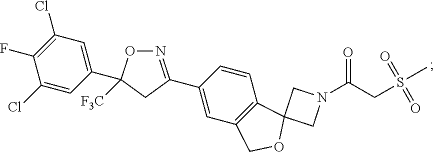 [SMILES: CS(=O)(=O)CC(=O)N1CC2(C1)OCc1cc(C3=NOC(c4cc(Cl)c(F)c(Cl)c4)(C(F)(F)F)C3)ccc12]

or a stereoisomer thereof, or a veterinarily acceptable salt thereof.

In another aspect of the invention, is a method for the treatment of a parasitic infection or infestation in a fish comprising administering to said fish an effective amount of a compound of Formula 1 that is the S-enantiomer of 1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone).

In yet another aspect of the invention, is a method for the treatment of a parasitic infection or infestation in a fish comprising administering to said fish an effective amount of a compound of Formula 1 that is the crystalline Form A of (S)-1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone).

In yet another aspect of the invention, is a method for the treatment of a parasitic infection or infestation in a fish comprising administering to said fish an effective amount of a compound of Formula 1 that is the amorphous S-enantiomer of (S)-1-(5′-5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone) prepared from the crystalline Form A of (S)-1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone).

Compounds of the present invention alone, or in combination with an additional veterinary agent(s) may be administered to a fish as (a) a single veterinary composition which comprises a compound of Formula (1), stereoisomer thereof, veterinarily acceptable salt thereof, and optionally, at least one additional veterinary agent and a veterinarily acceptable excipient, diluent, or carrier; or (b) two separate veterinary compositions comprising (i) a first composition comprising a compound of the present invention, stereoisomer thereof, veterinarily acceptable salt thereof, and a veterinarily acceptable excipient, diluent, or carrier, and (ii) a second composition comprising at least one additional veterinary agent and a veterinarily acceptable excipient, diluent, or carrier. The veterinary compositions may be administered simultaneously or sequentially and in any order.

The present invention provides a method of treating a parasitic infestation in a fish comprising administering an effective amount of any one of the foregoing compounds (hereinafter “the compound”) to said fish. In a more particular aspect of the invention, the parasitic infestation is an ectoparasite infestation. More particular still, the ectoparasite is a crustacean; specifically the crustacean is sea lice. In another aspect, the sea lice is at least one of Lepeophtheirus or Caligus species, specifically Lepeophtheirus salmonis, Caligus celmensi, Caligus curtus, Caligus dussumieri, Caligus elongates, Caligus longicaudatus, Caligus rogercresseyi, or Caligus stromii.

In another aspect of the invention, the fish is a farmed fish. In yet another aspect of the invention, the fish is selected from the group consisting of carp, tuna, tilapia, cod, halibut, trout or salmon. More particularly, the fish is salmon.

In another aspect of the invention, the compound is administered to the fish orally through a feed composition. In yet another aspect of the invention, the feed composition is a pellet comprising fat, nutrients, protein and the compound.

In another aspect of the invention, the compound is injected into the fish. More particularly, the compound is injected into the fish intraperitoneally (IP) or intramuscularly (IM).

In another aspect of the invention, the compound is co-administered with at least one of: another small molecule, an antigen, inactivated or killed virus or bacteria, or adjuvant.

In another aspect of the invention, the compound is administered to the fish by immersing the fish in a solution comprising the compound. In another embodiment, the compound is administered to the fish in a dose of at least 100 parts per billion (ppb).

In another aspect of the invention, the compound is co-administered to the fish with an additional antiparasitic agent. In another embodiment, the compound is administered to a plurality of fish.

In another aspect of the invention of the present invention provides a composition for oral administration to a fish comprising the compound and fish food. More particularly, the composition comprises fat, nutrients, protein and the compound. In another embodiment, the fish food comprises at least one of corn starch or oil. More particularly, the oil is vegetable oil or herring oil.

In yet another aspect of the invention, is a kit comprising the aforementioned composition and instructions for administration of the composition to fish.

In another aspect of the invention, the present invention is directed to the use of the compound in treating a sea lice infestation in a fish. In another aspect of the invention, the invention is directed to use of the compound in the preparation of a medicament for treating sea lice infestation in fish.

Other objects, features and advantages of the present invention will become apparent from the following detailed description. It should be understood, however, that the detailed description and the specific examples, while indicating embodiments of the invention, are given by way of illustration only, since various changes and modifications within the spirit and scope of the invention will become apparent to those skilled in the art from this detailed description.

### DETAILED DESCRIPTION

For purposes of the present invention, as described and claimed herein, the following terms and phrases are defined as follows:

“Additional veterinary agent(s)” as used herein, unless otherwise indicated, refers to other veterinary or pharmaceutical compounds or products that provide a therapeutically effective amount of said agents that are useful for the treatment of a parasitic infection in a fish.

“Alkoxy”, as used herein, unless otherwise indicated, refers to an oxygen moiety having a further alkyl substituent. The alkyl portion (i.e., alkyl moiety) of an alkoxy group has the same definition as below. Non-limiting examples include: —OCH₃, —OCH₂CH₃, and the like.

“Alkyl”, as used herein, unless otherwise indicated, refers to saturated monovalent hydrocarbon alkane radicals of the general formula CₙH₂ₙ₊₁. The alkane radical may be straight or branched and may be unsubstituted or substituted. For example, the term “(C₁-C₆)alkyl” refers to a monovalent, straight or branched aliphatic group containing 1 to 6 carbon atoms. Non-exclusive examples of (C₁-C₆) alkyl groups include, but are not limited to methyl, ethyl, propyl, isopropyl, sec-butyl, t-butyl, n-propyl, n-butyl, i-butyl, s-butyl, n-pentyl, 1-methylbutyl, 2-methylbutyl, 3-methylbutyl, neopentyl, 3,3-dimethylpropyl, 2-methylpentyl, hexyl, and the like. The alkyl moiety may be attached to the chemical moiety by any one of the carbon atoms of the aliphatic chain. Alkyl groups are optionally substituted as described herein. Further when used in compound words such as alkylphenyl, said alkyl moiety has the same meaning as herein defined and may be attached to the chemical moiety by any one of the carbon atoms of the aliphatic chain. Non-limiting examples of the compound word, alkylphenyl include: C₁alkylphenyl is —CH₂phenyl, C₂alkylphenyl is —CH₂CH₂phenyl, C₀phenyl is phenyl, and the like.

“Alkenyl” as used herein, unless otherwise indicated, refers to a straight or branched aliphatic hydrocarbon chain having 2- to 6-carbon atoms and containing at least one carbon-carbon double bond (for example —C═C—, or —C═CH₂). Non-exclusive examples of alkenyl include: ethenyl, 1-propenyl, 2-propenyl, isopropenyl, 1-butenyl, 2-butenyl, 3-butenyl, 2-pentenyl, and the like.

“Alkynyl” as used herein, unless otherwise indicated, refers to straight or branched aliphatic hydrocarbon chain having 2- to 6-carbon atoms and containing at least one carbon-carbon triple bond (for example, —C≡C— or —C≡CH). Non-exclusive examples of alkynyl include: ethynyl, 2-propynyl, 1-methyl-2-propynyl, 2-butynyl, 3-butynyl, 2-methyl-3-butynyl, and the like.

“Carbocyclic”, as used herein, unless otherwise indicated, refers to a partially saturated or saturated 5- to 7-membered ring containing only carbon atoms and can be monocyclic or part of a fused ring or spiro ring moiety. Examples of carbocyclic rings include cyclopentane, cyclohexane, and cycloheptane. The carbocyclic ring is optionally substituted as described herein.

“Chiral”, as used herein, unless otherwise indicated, refers to the structural characteristic of a molecule that makes it impossible to superimpose it on its mirror image, (e.g., “R” and “S” enantiomers). The term is also depicted as an asterisk (i.e.,*) in the Examples and preparations and refers to a chiral center which includes both the S and R enantiomers.

“Compounds of the present invention”, as used herein, unless otherwise indicated, refers to compounds of Formula (1), and stereoisomers thereof.

“Cycloalkyl”, as used herein, unless otherwise indicated, includes fully saturated or partially saturated carbocyclic alkyl moieties. Non-limiting examples of partially saturated cycloalkyls include: cyclopropene, cyclobutene, cycloheptene, cyclooctene, cyclohepta-1,3-diene, and the like. Preferred cycloalkyls are 3- to 6-membered saturated monocyclic rings including cyclopropyl, cyclobutyl, cyclopentyl, and cyclohexyl. The cycloalkyl group may be attached to the chemical moiety by any one of the carbon atoms within the carbocyclic ring. Cycloalkyl groups are optionally substituted with at least one substituent. Further when used in compound words such as alkylcycloalkyl, said alkyl and cycloalkyl moiety has the same meaning as herein defined and may be attached to the chemical moiety by any one of the carbon atoms of the aliphatic chain. Examples of C₀-C₆alkylC₃-C₆cycloalkyl include, methylcyclopropane (C₁alkylC₃cycloalkyl or —CH₂cyclopropane), ethylcyclopropane (C₂alkylC₃cycloalkyl or —CH₂CH₂cyclopropane), methylcyclobutane (C₁alkylC₄cycloalkyl or —CH₂cyclobutane), ethylcyclobutane (C₂alkylC₄cycloalkyl or —CH₂CH₂cyclobutane), methylcyclohexane (C₁alkylC₆cycloalkyl or —CH₂cyclohexane), and the like. C₀alkylC₃-C₆cycloalkyl is C₃-C₆cycloalkyl. Cycloalkyl moieties are optionally substituted as described herein.

“Fish” as used herein, unless otherwise indicated, refers to the taxonomic class Chondrichthyes (cartilaginous fishes, e.g., sharks and rays) and Osteichthyes (bony fishes) which live in water, have gills or mucus-covered skin for respiration, fins, and may have scales. Non-exclusive examples of fish include food fish, breeding fish and aquarium or pond fish of all ages occurring in freshwater, sea water and brackish water. The food fish and breeding fish include, for example, carp, eel, trout, whitefish, salmon, bream, roach, rudd, chub, sole, plaice, halibut, Japanese yellowtail (Seriola quinqueradiata), Japanese eel (Anguilla japonica), tuna, red sea bream (Pagurus major), sea bass (Dicentrarchus labrax), grey mullet (Mugilus cephalus), pompano, gilthread seabream (Sparus auratus), Tilapia spp., Cichlidae species such as plagioscion, channel catfish and “salmon”. Within the scope of this invention will be understood as comprising all representatives of the family Salmonidae, especially of the subfamily salmonini and, preferably, the following species: Salmo salar (Atlantic salmon); Salmo trutta (brown or sea trout); Salmon gairdneri (rainbow trout); and the Pacific salmon (Oncorhynchus): O. gorbuscha; O. keta; O. nekra; O. kisutch, O. tshawytscha and O. mason; also comprised are artificially propagated species such as Salvelinus species and Salmo clarkia.

In another aspect of the invention, the fish are kept in sea water tanks or cages. The cages are moored in sea inlets such that a constant flow of water passes through them in order to ensure a sufficient supply of oxygen. A constant flow of salt water in the sea water tanks is also maintained along with a supply of oxygen. In this artificial environment the fish are fed and, if necessary, provided with medication until they mature sufficiently for marketing as edible fish or are selected for further breeding.

Extremely intensive cage stocking is maintained in these fish farms. In this pure monoculture, the exceedingly high fish densities coupled with the other stress factors cause the caged fish to become in general markedly more susceptible to disease, epidemics and parasites than their free-living co-specifics. In order to maintain healthy populations, the caged fish must be treated regularly with bactericides and permanently monitored.

“Halogen” or “halo”, as used herein, unless otherwise indicated, refers to fluorine, chlorine, bromine and iodine. Further, when used in compound words such as “haloalkyl”, “haloalkoxy”, “haloalkenyl”, or “haloalkynyl”, said alkyl, alkoxy, alkenyl, and alkynyl may be partially or fully substituted with halogen atoms which may be the same or different and said alkyl, alkoxy, alkenyl, and alkynyl moiety has the same meaning as above and may be attached to the chemical moiety by any one of the carbon atoms of the aliphatic chain. Examples of “haloalkyl” include F₃C—, ClCH₂—, CF₃CH₂— and CF₃CCl₂—, and the like. The term “haloalkoxy” is defined analogously to the term “haloalkyl”. Examples of “haloalkoxy” include CF₃O—, CCl₃CH₂O—, HCF₂CH₂CH₂O— and CF₃CH₂O—, and the like. The term “haloalkenyl is defined analogously to the term “haloalkyl” except that the aliphatic chain contains at least one carbon-carbon double bond. Examples of “haloalkenyl” include CF₃C═C—, Cl₃CC═C—, HCF₂C═C— and CF₃C═CC—, and the like. The term “haloalkynyl” is defined analogously to the term “haloalkyl” except that the aliphatic chain contains at least one carbon-carbon triple bond. Examples of “haloalkynyl” include CF₃C≡C—, Cl₃CC≡C—, HCF₂C≡C— and CF₃C≡CC—, and the like.

“Heteroaryl” or “Het”, as used herein, unless otherwise indicated, refers to a 5- to 6-membered aromatic monocyclic ring or an 8- to 10-membered fused aromatic ring where said monocyclic- and fused-ring moiety contains one or more heteroatoms each independently selected from N, O, or S, preferably from one to four heteroatoms. Non-exclusive examples of monocyclic heteroaryls include pyrrolyl, furanyl, thiophenyl, pyrazolyl, imidazolyl, triazolyl, tetrazolyl, thiazolyl, isoxazolyl, oxazolyl, oxadiazolyl, thiadiazolyl, pyridinyl, pyridazinyl, pyrimidinyl, pyrazinyl, and the like. Non-exclusive examples of fused heteroaryls include: benzofuranyl, benzothiophenyl, indolyl, benzimidazolyl, indazolyl, benzotriazolyl, thieno[2,3-c]pyridine, thieno[3,2-b]pyridine, benzo[1,2,5]thiadiazole, and the like. The heteroaryl group may be attached to the chemical moiety by any one of the carbon atoms or nitrogen heteroatoms within the monocyclic or fused ring. Further when used in compound words such as alkylheteroaryl, said alkyl and heteroaryl moiety have the same meaning as herein defined and may be attached to the chemical moiety by any one of the carbon atoms of the aliphatic chain. For example, C₀alkylheteroaryl is heteroaryl, C₁alkylheteroaryl is —CH₂heteroaryl, C₀alkylheteroaryl is —CH₂CH₂heteroaryl, and the like. Heteroaryls are optionally substituted as described herein.

“Heterocycle”, as used herein, unless otherwise indicated, refers to a partially saturated or saturated 3- to 7-membered monocyclic ring containing one or more heteroatoms each independently selected from N, O, or S, preferably from one to four heteroatoms. The heterocyclic ring can be part of a fused ring or spiro-ring moiety. Non-exclusive examples of heterocycle include oxirane, thiarane, aziridine, oxetane, azetidine, thiatane, tetrahydrofuran, tetrahydrothiophene, pyrrolidine, tetrahydropyrane, piperidine, piperazine, tetrahydropyridine, 2H-azirine, 2,3-dihydro-azete, 3,4-dihydro-2H-pyrrole, and the like. The heterocycle group may be attached to the chemical moiety by any one of the carbon atoms or nitrogen heteroatoms within the ring. Further when used in compound words such as alkylheterocycle, said alkyl and heterocycle moiety have the same meaning as herein defined and may be attached to the chemical moiety by any one of the carbon atoms of the aliphatic chain. For example, C₀alkylheterocycle is heterocycle, C₁alkylheterocycle is —CH₂heterocycle, C₀alkylheterocycle is —CH₂CH₂heterocycle, and the like. Heterocycles are optionally substituted as described herein.

“Optionally substituted”, is used herein interchangeably with the phrase substituted or unsubstituted. Unless otherwise indicated, an optionally substituted group may have a substituent at each substitutable position of the group, and each substitution is independent of the other. An optionally substituted group also may have no substituents. Therefore, the phrase “optionally substituted with at least one substituent” means that the number of substituents may vary from zero up to a number of available positions for substitution.

“Parasite(s)”, as used herein, unless otherwise indicated, refers to endoparasites and ectoparasites. Endoparasites are parasites that live within the body of its host and include helminths (e.g., trematodes, cestodes, and nematodes) and protozoa. Ectoparasites are organisms of the Arthropoda phylum (e.g., arachnids, insects, and crustaceans (e.g., copepods-sea lice) which feed through or upon the skin of its host. Preferred arachnids are of the order Acarina, e.g., ticks and mites. Preferred insects are midges, fleas, mosquitos, biting flies (stable fly, horn fly, blow fly, horse fly, and the like), bed bugs, and lice. Preferred compounds of the present invention can be used for the treatment of parasites, i.e., treatment of a parasitic infection or infestation.

“Sea lice” as used herein are parasitic crustaceans within the order Siphonostomatoida, family Caligidae. Two representatives of the class cause substantial losses in yield: Lepeophtheirus and Caligus. Lepeophtheirus has a brown, horseshoe-shaped carapace and Caligus is also brown, but smaller. Species within Lepeophtheirus include Lepeophtheirus salmonis and within Caligus include Caligus celmensi, Caligus curtus, Caligus dussumieri, Caligus elongates, Caligus longicaudatus, Caligus rogercresseyi and Caligus stromii.

These sea lice injure the fish by feeding on the scales, epithelium and the mucosa. When infestation is severe, these parasites also damage underlying dermis. If, moreover, infected salmon are kept in cooler waters, then they are normally no longer able to protect themselves from these pests. As a consequence, secondary infections and water-logging will occur, even if the sea lice are removed. In extreme cases, severe wounding resulting from infestation by these parasites leads to further tissue damage caused by ultraviolet radiation or to the death of the fish from osmotic shock or the secondary infections.

“Therapeutically effective amount”, or “effective amount” as used herein, unless otherwise indicated, refers to an amount of the compounds of the present invention that (i) treat the particular parasitic infection or infestation, (ii) attenuates, ameliorates, or eliminates one or more symptoms of the particular parasitic infection or infestation, or (iii) prevents or delays the onset of one or more symptoms of the particular parasitic infection or infestation described herein.

“Treatment”, “treating”, and the like, as used herein, unless otherwise indicated, refers to reversing, alleviating, preventing or inhibiting the parasitic infection, infestation, or condition. As used herein, these terms also encompass, depending on the condition of the fish, preventing the onset of a disorder or condition, or of symptoms associated with a disorder or condition, including reducing the severity of a disorder or condition or symptoms associated therewith prior to affliction with said infection or infestation. Thus, treatment can refer to administration of the compounds of the present invention to a fish that is not at the time of administration afflicted with the infection or infestation. Treating also encompasses preventing the recurrence of an infection or infestation or of symptoms associated therewith as well as references to “control” (e.g., kill, repel, expel, incapacitate, deter, eliminate, alleviate, minimize, and eradicate).

Reference to treating a parasitic infestation “in” a fish is understood to constitute treatment of an external parasite, such an ectoparasite, which feeds “on” a fish and not necessarily exist inside the fish.

“Veterinary acceptable” as used herein, unless otherwise indicated, indicates that the substance or composition must be compatible chemically and/or toxicologically, with the other ingredients comprising a formulation, composition, and/or the fish being treated therewith. The term “pharmaceutically” acceptable has the same meaning as that recited for “veterinarily” acceptable.

The compositions disclosed can be administered in a variety of ways. It should be noted that the composition can be administered alone or in combination with one or more pharmaceutically acceptable carriers, stabilizers, preservatives, colorants, flavorants, and excipients.

Reference to “fish food” indicates substances specially adapted for administration to fish. Particularly, at least one of fats, nutrients, protein, vitamins or carbohydrates in flake or pellet form, which is capable of adsorbing or mixing with the active compound(s) of the present invention. Preferably, the fish food includes corn starch, vegetable oil and/or fish oil, such as herring oil.

The compositions disclosed can be formulated with conventional carriers and excipients, which are selected in accord with ordinary practice. Aqueous formulations are preferably prepared in sterile form, and when intended for delivery by routes other than oral administration, generally are isotonic. Excipients include ascorbic acid and other antioxidants, chelating agents (e.g., EGTA and EDTA), carbohydrates (e.g., dextrin), hydroxyalkylcellulose, hydroxyalkylmethylcellulose, stearic acid, and the like. The pH of the formulations ranges from about 3 to about 11.

Examples of physiologically acceptable carriers for routes of administration other than oral administration include but are not limited to saline solutions (e.g., normal saline, Ringer's solution, PBS (phosphate-buffered saline); polysorbate 80; L-arginine; polyvinylpyrrolidone; α-D-glucopyranosyl; α-D-glucopyranoside (trehalose); and combinations, thereof. For example, trehalose can be present in the composition in an amount from about 2 to about 10% weight/volume of the composition. In another example, when trehalose and polysorbate 80 are both present in the composition, trehalose can be present in the amount of about 4 to about 6% wt./vol. and the polysorbate 80 can be present in the amount of about 0.001 to 0.01% (wt./vol.) and generally mixtures of various physiologically compatible salts including potassium and phosphate salts with or without sugar additives (e.g., glucose).

Suitable excipients for use in the immunogenic formulations are, for example, water, saline, dextrose, glycerol, and ethanol. Non-toxic auxiliary substances, such as wetting agents, buffers, stabilizers, or emulsifiers can also be added to the composition.

Parenteral administration, if used, is generally characterized by injection. Sterile injectables can be prepared in conventional forms, either as liquid solutions or suspensions, solid forms suitable for solution or suspension in liquid prior to injection, or as emulsions.

For each recipient, the total amount of the composition necessary for administration can be derived by routine practice of those skilled in the art. The exact amount of such compositions required may vary from fish to fish or stock to stock.

The formulations include those suitable for the foregoing administration routes. The formulations can conveniently be presented in unit dosage form and can be prepared by any of the methods well known in the art of veterinary science. In general, the formulations are prepared by uniformly and intimately bringing into association the active ingredient with liquid carriers or finely divided solid carriers or both, and then, if necessary, shaping the product.

The oil phase of the emulsions of this invention can be constituted from known ingredients in a known manner. While the phase can comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier, which acts as a stabilizer. It is also preferred to include both an oil and a fat.

Veterinary carriers are materials useful for the purpose of administering the composition and can be solid, liquid or gaseous materials, which are otherwise inert or acceptable in the veterinary art and are compatible with the active ingredient. These veterinary compositions can be administered orally, parenterally, or by any other desired route.

As indicated above, the present disclosure provides compositions and methods that employ the compound administered orally. In certain embodiments of the present disclosure, the compound is used in the form of a pure powder, though other formulations may be appropriate. The oral feed compositions and formulations which follow are all contemplated in the fish food of the present invention.

Exemplary oral non-toxic inert suitable excipients include, for example, fillers and extenders, binders, humectants, solution retarders, absorption accelerators, wetting agents, adsorbents or lubricants, which may have a solid, semisolid or liquid consistency. Such excipients are known to those of skill in the art.

The compound may be added to the feed by customary methods, by simply mixing as a pure substance, such as a powder, or in a formulated form mixed with edible, nontoxic excipients in the form of a premix. Thus, the compound may be formulated together with pharmaceutically active compounds, minerals, salts, elements, vitamins, proteins, fats, colorants and/or flavorings.

It will be understood that the amount of the compound that is administered to a fish to achieve the desired effect can be substantially varied because of the favorable non-toxic properties of the compound. In one embodiment, the compound is administered orally at about 0.005 to 5000 mg/kg, in particular 0.01 to 500 mg/kg (i.e. mg compound per kg fish body weight per day). Moreover, the compound can be administered at relatively high doses, such as exceeding (i.e. greater than) 0.01 mg/kg, 0.1 mg/kg, 1 mg/kg, 10 mg/kg or even greater than 100 mg/kg. The duration of administration can be from a few hours or days up to several years. When applied topically in water baths or orally, the compound can be present, for example, in a concentration by weight of about 0.0005 to 50, in particular 0.001 to 10 ppm, thus greater than 100 parts per billion in one embodiment.

All conventional or special feed compositions can be used, and these preferably contain the customary balance of energy carriers and builders, which are necessary for a balanced diet, including vitamins and minerals. For example, the feed can be composed of vegetable materials, for example, hay, roots, cereals, cereals by-products, kelp, lettuce, animal materials, for example meat, fats, bone meal, fish products, vitamins, for example vitamin A, D complex and B complex, proteins, amino acids, for example DL-methionine and inorganic substances, for example lime and sodium chloride.

Feed concentrates contain the active compound in addition to edible substances, for example rye meal, corn meal, corn starch, soy bean meal or lime, where appropriate with other nutrients and builders, and proteins, mineral salts and vitamins. They can be produced by the customary mixing methods.

When formulated as a feed, the compound may be admixed with one or more fish-appropriate feedstuff. Alternatively or additionally, the premix may comprise other nontoxic material(s), which are typically though not exclusively carbohydrate-based, and are of sufficient granularity to facilitate thorough mixing when added to larger quantities of feedstuff. Other nutrients, proteins, mineral salts, and vitamins may be included in the compound premix.

The feed mixtures indicated are adjusted to be appropriate preferably for the rearing, fattening and harvesting of fish. When using a pre-mix of concentrated compound, it is generally then added to additional stores of untreated food. The optimum final concentration of the compound will depend upon the amount of food to be consumed by the fish and can be readily determined by those of skill in the art. The type of food and its composition will be determined by the skilled artisan based upon the particular requirements of the species of fish and location or size

In a preferred embodiment fish food can be combined with the compound to form of a pellet for oral administration to the fish. The pellet may include ingredients such as corn starch, oil, such as herring oil or vegetable oil, and the compound. The compound can be added by surface coating of fish feed pellets or coextrusion with fish meal ingredients to form pellets. In surface coating, a premix is typically suspended in fish oil and the suspension is poured onto the feed under mixing in a suitable mixer (ribbon or cement type mixer). A premix comprising the compound also be dusted onto feed pellets followed by the oil coat, which is referred to as the double-coating procedure. In a co-extrusion method, a premix of the compound is blended with feed ingredients in a mixer. The blend is then conditioned and passed through an extruder under high heat and humidity conditions. The extruded pellets are then dried and coated with oil, if desired.

Additional aquaculture formulation techniques and compositions are described in Z. J. Shao, Advanced Drug Delivery Reviews, 50 (2001) 229 243, the contents of which is hereby incorporated by reference as if set forth fully herein.

Examples of various formulations/compositions for use in the present invention are provided as follows:

A. Emulsifiable Concentrates:
Active compound: 1 to 90%, preferably 5 to 20%, surfactant: 1 to 30%, preferably 10 to 20% solvent: 5 to 98%, preferably 70 to 85%

B. Suspension Concentrates:
Active compound: 5 to 75%, preferably 10 to 50%, water: 94 to 24%, preferably 88 to 30%, surfactant: 1 to 40%, preferably 2 to 30%

C. Wettable Powders:
Active compound: 0.5 to 90%, preferably 1 to 80% surfactant: 0.5 to 20%, preferably 1 to 15% solid carrier: 5 to 99%, preferably 15 to 98%

D. Granulates:
Active compound: 0.5 to 30%, preferably 3 to 15%, solid carrier: 99.5 to 70%, preferably 97 to 85%

E. Emulsifiable Concentrates
Active compound: 25%-50%; calcium dodecylbenzene sulfonate 5%-8%; castor oil polyethylene glycol ether 5%; tributylphenol polyethylene glycol 4%-12%; ether cyclohexanone; 15%-20%; and xylene mixture 20%-65%

F. Extruder Granulate
Active compound: 10%, sodium ligninsulfonate 2%, carboxymethyl cellulose 1%, kaolin 87%. Active compound is mixed with the adjuvants and the mixture is ground and moistened with water. This mixture is extruded, granulated and then dried in a stream of air.

G. Coated Granulates
Active compound 3%, polyethylene glycol 3%, kaolin 94%. The finely ground active substance is uniformly applied, in a mixer, to the kaolin moistened with polyethylene glycol. Non-dusty coated granulates are obtained in this manner.

H. Suspension Concentrate
Active compound 40%, ethylene glycol 10%, nonylphenol polyethylene glycol ether 6%, sodium ligninsulfonate 10%, carboxymethyl cellulose 1%, 37% aqueous formaldehyde solution 0.2%, silicone oil in the form of a 75%, 0.8% aqueous emulsion water 32%. The finely ground active substance is homogeneously mixed with the adjuvants, giving a suspension concentrate from which suspensions of any desired concentration can be obtained by dilution with water.

I. Injection Formulations
Ampoule containing active compound, Disodium Pamidronat Pentahydrate and Water. After Dissolution (Concentration 3 Mg/Ml), the Solution can be Used for Injections. Active compound 15.0 mg, mannitol 250 mg, water for injection 5 ml.

J. Pellet Formulation
Derquatel 1-20%, herring or vegetable oil 1-5%, corn starch q.s. to reach 100% (about 75%-98%). Ingredients are mixed into a pellet formulation with the oil acting as an adherent and flavorant.

The compounds and compositions can also be used in combination with other active ingredients. Such combinations are selected based on the condition to be treated, cross-reactivities of ingredients, and pharmacological properties of the combination. For instance, multifunctional agents, such as polyvalent vaccines are preferable in fish treatment, thus the composition may be administered with antigens targeting other diseases. These compounds and compositions can be administered together with, or in the same course of, therapy with the compounds and compositions described herein. The individual components of the combination can be administered either sequentially or simultaneously in separate or combined veterinary formulations.

In another aspect of the invention, is the use of crystalline polymorphic Form A of (5)-1-(5-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone as an antiparasitic for fish.

The characteristic powder x-ray diffraction peaks of Form A expressed in degrees 2θ (±0.2° θ), interplanar spacings (d), and respective intensities (%) are displayed in Table 1 below.

TABLE 1
PXRD Polymorphic Form A (S-enantiomer)

| | | | Intensity |
|---|---|---|---|
| Peak | 2-Theta° | d-spacing | (%) |
| 1 | 3.98 | 22.16 | 3.3 |
| 2 | 4.25 | 20.76 | 3 |
| 3 | 4.70 | 18.79 | 38.8 |
| 4 | 5.13 | 17.20 | 7.2 |
| 5 | 5.24 | 16.84 | 6.5 |
| 6 | 5.52 | 15.99 | 7.3 |
| 7 | 5.98 | 14.76 | 16.6 |
| 8 | 9.39 | 9.42 | 36.1 |
| 9 | 11.61 | 7.62 | 28 |
| 10 | 13.26 | 6.67 | 16.4 |
| 11 | 13.72 | 6.45 | 20.4 |
| 12 | 14.10 | 6.28 | 33.4 |
| 13 | 14.52 | 6.10 | 18.5 |
| 14 | 14.94 | 5.92 | 27.4 |
| 15 | 15.70 | 5.64 | 33.3 |
| 16 | 16.13 | 5.49 | 16.4 |
| 17 | 16.60 | 5.34 | 29.2 |
| 18 | 17.18 | 5.16 | 60 |
| 19 | 18.06 | 4.91 | 23.4 |
| 20 | 18.29 | 4.85 | 22.9 |
| 21 | 18.51 | 4.79 | 24.7 |
| 22 | 18.83 | 4.71 | 58.8 |
| 23 | 19.12 | 4.64 | 33.7 |
| 24 | 19.32 | 4.59 | 29.6 |
| 25 | 19.72 | 4.50 | 23.7 |
| 26 | 20.07 | 4.42 | 100 |
| 27 | 20.97 | 4.23 | 33.3 |
| 28 | 21.42 | 4.14 | 55.1 |
| 29 | 22.03 | 4.03 | 30.5 |
| 30 | 22.54 | 3.94 | 42.8 |
| 31 | 22.76 | 3.90 | 26.1 |
| 32 | 23.62 | 3.76 | 39.5 |
| 33 | 24.21 | 3.67 | 24.9 |
| 34 | 24.61 | 3.61 | 22.7 |
| 35 | 25.26 | 3.52 | 18.5 |
| 36 | 25.91 | 3.44 | 25.6 |
| 37 | 27.01 | 3.30 | 24.2 |
| 38 | 27.57 | 3.23 | 20.5 |
| 39 | 28.02 | 3.18 | 18.9 |
| 40 | 28.42 | 3.14 | 53.5 |
| 41 | 29.20 | 3.06 | 21.7 |
| 42 | 29.69 | 3.01 | 20.6 |
| 43 | 30.13 | 2.96 | 22.2 |
| 44 | 31.36 | 2.85 | 18.2 |
| 45 | 31.70 | 2.82 | 18.3 |
| 46 | 32.65 | 2.74 | 17.4 |
| 47 | 33.59 | 2.67 | 20.6 |
| 48 | 34.25 | 2.62 | 19 |
| 49 | 35.24 | 2.54 | 18.4 |
| 50 | 36.35 | 2.47 | 19 |
| 51 | 37.13 | 2.42 | 25 |
| 52 | 37.51 | 2.40 | 18.6 |
| 53 | 38.27 | 2.35 | 18.7 |
| 54 | 39.01 | 2.31 | 18.3 |
| 55 | 40.69 | 2.22 | 21 |
| 56 | 41.08 | 2.20 | 19.4 |
| 57 | 43.2 | 2.09 | 19 |
| 58 | 45.59 | 1.99 | 17.5 |

In another aspect of the invention, Form A has characteristic PXRD peaks expressed in degrees 2θ (±0.2° θ) at about one or more of the following positions: 9.39, 14.10, 17.18, 18.83, 19.12, 20.07, 21.42, 22.54, 23.62, and 28.42, all of which have a relative intensity of at least 33%. In yet another aspect, Form A has a PXRD peak expressed in degrees 2θ (±0.2° θ) at about 20.07. In another aspect, Form A has a PXRD peak expressed in degrees 2θ (±0.2° θ) at about 20.07 and further comprises at least one additional diffraction peak expressed in degrees 2θ (±0.2° θ) selected from the group consisting of peaks at about 9.39, 14.10, 17.18, 18.83, 19.12, 20.07, 21.42, 22.54, 23.62, and 28.42. In another aspect of the present invention, Form A has characteristic PXRD peaks expressed in degrees 2θ (±0.2° θ) at about one or more of the following positions: 17.18, 18.83, 20.07, 21.42, 22.54, and 28.42, all of which have a relative intensity of at least 40%. In another aspect of the present invention, Form A has characteristic PXRD peaks expressed in degrees 2θ (±0.2° θ) at about one or more of the following positions: 17.18, 18.83, 20.07, 21.42, and 28.42, all of which have a relative intensity of at least 50%.

In another aspect of the present invention, Form A also exhibits a Fournier-Transform Infrared (FT-IR) spectrum at the 1800 to 600 cm⁻¹ range substantially as shown in Table 2. Characteristic FT-IR peaks of Form A are shown in Table 2 below.

TABLE 2
FT-IR of Polymorphic Form A

| Peak (cm⁻¹) | Absolute Intensity | Relative Intensity | Width |
|---|---|---|---|
| 1662 | −0.400 | 0.482 | 15.26 |
| 1459 | −0.148 | 0.219 | 26.81 |
| 1352 | −0.048 | 0.100 | 10.85 |
| 1304 | −0.432 | 0.523 | 11.36 |
| 1191 | −0.334 | 0.390 | 37.15 |
| 1166 | −0.285 | 0.075 | 7.49 |
| 1133 | −0.164 | 0.148 | 13.91 |
| 1023 | −0.118 | 0.189 | 22.58 |
| 984 | −0.011 | 0.065 | 13.26 |
| 912 | −0.180 | 0.258 | 17.62 |
| 815 | −0.131 | 0.199 | 36.74 |
| 757 | −0.046 | 0.117 | 10.61 |
| 721 | −0.025 | 0.085 | 14.90 |
| 659 | −0.043 | 0.130 | 9.62 |
| 625 | −0.018 | 0.081 | 12.07 |

In another aspect of the present invention, Form A exhibits characteristic Fournier-Transform Infrared (FT-IR) peaks at the 1800 to 600 cm⁻¹ spectrum range at one or more of the following: 1662, 1459, 1352, 1304, 1191, 1166, 1133, 1023, 984, 912, 815, 757, 721, 659, and 625 cm⁻¹.

In another aspect of the present invention, Form A also exhibits a differential scanning calorimetry (DSC) thermogram substantially characterized by a predominant endotherm peak at about 145.53° C. In another embodiment of the present invention, Form A also exhibits a differential scanning calorimetry (DSC) thermogram, which is characterized by a predominant endotherm peak at about 145.53° C. with an onset peak at about 135.26° C.

In another aspect of the present invention, Form A is characterized by PXRD peaks expressed in degrees 2θ (±0.2° θ) at one or more of the following positions: 9.39, 14.10, 17.18, 18.83, 19.12, 20.07, 21.42, 22.54, 23.62, and 28.42 and with characteristic FT-IR peaks at the 1800 to 600 cm⁻¹ spectrum range at one or more of the following 1662, 1459, 1352, 1304, 1191, 1166, 1133, 1023, 984, 912, 815, 757, 721, 659, and 625 cm⁻¹. In another aspect of the invention, Form A is characterized by PXRD peaks expressed in degrees 2θ (±0.2° θ) at one or more of the following positions: 9.39, 14.10, 17.18, 18.83, 19.12, 20.07, 21.42, 22.54, 23.62, and 28.42 and with a predominant DSC endotherm peak at about 145.53° C. In yet another aspect of the invention, Form A is characterized by FT-IR peaks at the 1000 cm⁻¹ spectrum range at one or more of the following 1662, 1459, 1352, 1304, 1191, 1166, 1133, 1023, 984, 912, 815, 757, 721, 659, and 625 cm⁻¹, and with a DSC endotherm peak at about 145.53° C.

In yet another aspect of the invention, Form A is characterized by PXRD peaks expressed in degrees 2θ (±0.2° θ) at one or more of the following positions: 9.39, 14.10, 17.18, 18.83, 19.12, 20.07, 21.42, 22.54, 23.62, and 28.42, characteristic FT-IR peaks at the 1800 to 600 cm⁻¹ spectrum range at one or more of the following 1662, 1459, 1352, 1304, 1191, 1166, 1133, 1023, 984, 912, 815, 757, 721, 659, and 625 cm⁻¹, and with a predominant DSC endotherm peak at about 145.53° C.

In another aspect of the present invention, Form A has characteristic PXRD peaks expressed in degrees 2θ (±0.2° θ) at about one or more of the following positions: 17.18, 18.83, 20.07, 21.42, 22.54, and 28.42, and with characteristic FT-IR peaks at the 1800 to 600 cm⁻¹ spectrum range at one or more of the following 1662, 1459, 1352, 1304, 1191, 1166, 1133, 1023, 984, 912, 815, 757, 721, 659, and 625 cm⁻¹.

In another aspect of the present invention, Form A has characteristic PXRD peaks expressed in degrees 2θ (±0.2° θ) at about one or more of the following positions: 17.18, 18.83, 20.07, 21.42, 22.54, and 28.42, and with a DSC endotherm peak at about 145.53° C.

In another aspect of the present invention, Form A has characteristic PXRD peaks expressed in degrees 2θ (±0.2° θ) at about one or more of the following positions: 17.18, 18.83, 20.07, 21.42, 22.54, and 28.42, with characteristic FT-IR peaks at the 1800 to 600 cm⁻¹ spectrum range at one or more of the following 1662, 1459, 1352, 1304, 1191, 1166, 1133, 1023, 984, 912, 815, 757, 721, 659, and 625 cm⁻¹, and with a DSC endotherm peak at about 145.53° C.

In another aspect of the present invention, Form A has characteristic PXRD peaks expressed in degrees 2θ (±0.2° θ) at about one or more of the following positions: 17.18, 18.83, 20.07, 21.42, and 28.42, and with characteristic FT-IR peaks at the 1800 to 600 cm⁻¹ spectrum range at one or more of the following 1662, 1459, 1352, 1304, 1191, 1166, 1133, 1023, 984, 912, 815, 757, 721, 659, and 625 cm⁻¹.

In another aspect of the present invention, Form A has characteristic PXRD peaks expressed in degrees 2θ (±0.2° θ) at about one or more of the following positions: 17.18, 18.83, 20.07, 21.42, and 28.42, and with a DSC endotherm peak at about 145.53° C.

In another aspect of the present invention, Form A has characteristic PXRD peaks expressed in degrees 2θ (±0.2° θ) at about one or more of the following positions: 17.18, 18.83, 20.07, 21.42, and 28.42, and with characteristic FT-IR peaks at the 1800 to 600 cm⁻¹ spectrum range at one or more of the following 1662, 1459, 1352, 1304, 1191, 1166, 1133, 1023, 984, 912, 815, 757, 721, 659, and 625 cm⁻¹, and with a DSC endotherm peak at about 145.53° C.

In another aspect of the present invention, Form A also exhibits a differential scanning calorimetry (DSC) thermogram, which displays four different Form A samples, which is characterized by a predominant endotherm peak at about 144.01, 144.82, 146.32, and 146.92° C. with onset peaks at about 133.95, 136.29, 137.54, and 137.96° C. On average, the DSC thermogram of the four samples is characterized by a predominant endotherm peak at about 145.52° C. with an onset peak at about 136.44° C.

Compounds of Formula (1) for use in the compositions and methods of the present invention, particularly, 1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone, include the racemate, (S)-enantiomer, the crystalline Form A, as well as the amorphous (S)-enantiomer prepared from the crystalline Form A, thereof.

Compounds for use in the compositions and methods of the present invention may be synthesized by synthetic routes that include processes analogous to those well known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, “Reagents for Organic Synthesis”, 1; 19, Wiley, New York (1967, 1999 ed.), or Beilsteins Handbuch der organischen Chemie, 4, Aufl. ed. Springer-Verlag, Berlin, including supplements (also available via the Beilstein online database)). For illustrative purposes, the reaction schemes depicted below demonstrate potential routes for synthesizing compounds of the present invention, and key intermediates. For a more detailed description of the individual reaction steps, see the Examples section below. A skilled artisan will appreciate that other suitable starting materials, reagents, and synthetic routes may be used to synthesize the compounds of the present invention and a variety of derivatives thereof. Further, many of the compounds prepared by the methods described below can be further modified in light of this disclosure using conventional chemistry well known to the skilled artisan.

Compounds of the present invention described herein contain at least one asymmetric or chiral center; and, therefore, exist in different stereoisomeric forms. The R and S configurations are based upon knowledge of known chiral inversion/retention chemistry. Unless specified otherwise, it is intended that all stereoisomeric forms of the compounds of the present invention as well as mixtures thereof, including racemic mixtures and diastereomeric mixtures, form part of the present invention.

Enantiomeric mixtures can be separated into their individual enantiomers on the basis of their physical chemical differences by methods well known to those skilled in the art, such as chromatography and/or fractional crystallization. A more detailed description of techniques that can be used to resolve stereoisomers of compounds from their racemic mixture can be found in Jean Jacques Andre Collet, Samuel H. Wilen, Enantiomers, Racemates and Resolutions, John Wiley and Sons, Inc. (1981).

Compounds of this invention can exist as one or more stereoisomers. The various stereoisomers include enantiomers, diastereomers and atropisomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereo isomers or as an optically active form.

Detailed schemes and synthetic routes for preparation of the compounds of the present invention are provided in WO2012/120399 and PCT/US2013/56945. In particular, the synthetic routes provided in Preparation 1-8 and the following Examples, particularly Example of the aforementioned patent application describe preferred routes of administration of the compounds described herein.

Particular aspects of the present invention are illustrated by the following Examples. It is to be understood, however, that the invention is not limited to the specific details of these Examples, as other variations thereof will be known, or apparent in light of the instant disclosure, to one of ordinary skill in the art.

### EXAMPLES

### Example 1

### 1-(5′-5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone

The “*” represents the chiral carbon.

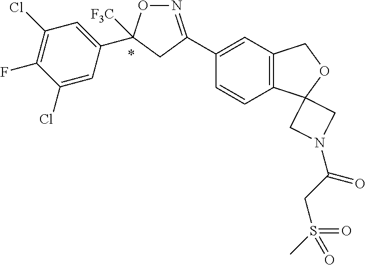 [SMILES: CS(=O)(=O)CC(=O)N1CC2(C1)OCc1cc(C3=NOC(c4cc(Cl)c(F)c(Cl)c4)(C(F)(F)F)C3)ccc12]

The p-toluenesulfonic acid salt of chiral-5′-5-(3,5-dichloro-4-fluorophenyl)-5-(trifluoromethyl)-4,5-dihydroisoxazol-3-yl)-3′H-spiro[azetidine-3,1′-isobenzofuran] (Preparation 7,157 g, 248 mmol) was stirred as a slurry in methyl tert-butyl ether (700 mL) at ambient temperature. To this was added 0.5N aqueous sodium hydroxide (600 mL, 300 mmol) and the mixture was stirred for 15 minutes at which time the two layers were clear. The aqueous layer was separated and the organics were washed with saturated brine (200 mL) and dried with sodium sulfate (5 grams). The organics were filtered to remove the solids.

In a separate flask, 43.2 gm (297 mmol) of 2-methansulfonylacetic acid was dissolved in DMF (300 mL) at ambient temperature. Carbonyldiimidazole (45.1 gm, 271 mmol) was added portion wise to the solution over 15 minutes to control foaming. After addition, the solution was stirred for 15 minutes at ambient temperature. The above ethereal solution of the amine was added to this reaction in one portion. The resulting solution was stirred at ambient temperature for 30 minutes. Water (800 mL) was added to quench the reaction. After stirring for two minutes, the aqueous layer is settled and removed. The organic layer is stirred at ambient temperature for one hour. During this time, the racemate precipitated from the reaction mixture. The mixture is then filtered through filter aid (Celite 545) to remove the racemic material. The sulfonamide remaining in solution is greater than 99% of a single isomer (i.e., S). The organic solution is washed with water twice (2×1 L) and concentrated to an off-white solid. (138.2 gm, 96%) Residual color can be removed by dissolving material in ethanol, stirring with 10 wt % charcoal (Darco G-60), filtering, and concentrating to a solid The asterisk (*) depicts a chiral center.

Alternatively, the racemate of the besylate salt of the sulfonamide can be removed by mixing methanesulfonylacetic acid (0.615 g, 1.3 eq) with the sulfonamide (2.1 g) in 9.3 mL ethyl acetate (EtOAc). Triethylamine is added dropwise over 1 minute (0.825 g, 2.4 eq) at about 18-22° C. The addition funnel is rinsed with 0.5 mL EtOAc and the resulting mixture is stirred for 30 minutes and cooled to <10° C. To this mixture, 4.313 g n-propylphosphonic anhydride (50 weight % in EtOAc), 2.0 eq) is added dropwise over 15 minutes at <10° C. The addition funnel is rinsed again with 1.5 mL EtOAc. The reaction mixture is warmed to 35° C. and stirred overnight. (UPLC >97% with <1% starting material). To the reaction was added 1.0 g Celite filter aid (50% loading) and filtered through a 1 g celite plug in a 15 mL coarse frit glass funnel (1.75 minute filtration) and rinsed with 4 mL EtOAc (2×). Chiral HPLC 98.8% S enantiomer, 1.2% R; UPLC >97%, filtrate volume=18 mL).

### Example 2

### Preparation of crystal Form A of 1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone (S-enantiomer)—Crystallization of the Amorphous S-Enantiomer

Originally, Form A seeds were prepared by dissolving between 100 mg and 200 mg of the amorphous S form of 1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone in methanol at room temperature. A small aliquot of this solution was placed in an uncapped 4-mL glass vial. The 4-mL glass vial was placed inside a larger 20-mL amber vial containing several mL of diisopropyl ether and then the 20 mL vial was capped. The solvent vapors were allowed to evaporate/diffuse for a period of 5 days, at which time solids were noted. Examination of the solids under light microscopy revealed highly birefringent crystalline particles. Analysis by hot stage microscopy demonstrated a melting point between 130 and 170° C. Further examination confirmed Form A, (e.g., the seed crystals).

Form A can be prepared by charging 15.4 grams of the amorphous S enantiomer described above, dissolved in 92 mL ethanol and 7.7 mL ethyl acetate, to a preheated 1-L jacketed reactor equipped with overhead stirring, temperature probe/readout, programmable chiller, nitrogen headspace purge, and water-cooled overhead condenser. Next, 54 mL of n-heptane is added. The resulting system is heated to 60° C. and a solution results. The solution is cooled to 45° C. over 15 minutes, and a hazy or milky solution results, without any signs of the formation of crystalline solids. 308 mg of polymorphic Form A that was hand ground with a mortar and pestle is then added. The seeds persist in the reactor. The system is held at 45° C., then the contents of the reactor are cooled to 30° C. at 1.5° C. per hour linearly, then cooled to 10° C. over three hours linearly, then held at 10° C. for 4.5 hours. A white, stirrable slurry results. The slurry is cooled to 0-1° C. over 20 minutes and held overnight (about 23 hours) at 0-1° C. The contents of the reactor are transferred to a sintered glass filter and vacuum is applied until a solid cake is observed. The cake is washed on the filter with about 40 mL of 60% n-heptane/40% ethanol denatured with 0.5% toluene. The cake is further washed washed with approximately 300 mL n-heptane. The cake is dried with air by pulling vacuum for about 1 hour, then the cake is dried further in a vacuum oven overnight at 40° C. The resulting 9.26 grams of Form A of 1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone were confirmed by DSC.

Alternatively, Form A can be prepared by charging 4 grams of the amorphous S form (isolated by rotavapping to a foam) to a 50-mL MultiMax reactor equipped with jacketed heating/cooling, overhead stirring, thermocouple, and a dispensing box. Add 24 mL of a solvent mixture consisting of 60 volume % ethanol (denatured with 0.5 volume % toluene), 35% heptane, and 5% ethyl acetate. Heat the mixture to 60° C., and a clear solution results. Cool to 45° C. over 20 minutes, then add seeds of polymorphic Form A (approximately 40 mg). The seeds persist in the reactor vessel. Hold for 2 hours at 45° C., then cool to 20° C. over 12.5 hours (about 2° C./hour) linearly, then hold at 20° C. while adding heptane (16 mL) over 1 hour using the dispensing box. At this point, a white slurry is observed. Then hold 1 hour at 20° C., and subsequently cool to −10° C. over 10 hours and hold at −10° C. for 3 hours. Filter the resulting slurry on a pre-chilled, sintered glass filter, and then wash with 10 mL of 80% heptane/20% ethanol (denatured with 0.5 vol % toluene), pre-chilled to approximately 0° C. Dry the cake in the vacuum oven over 2.5 days at 30° C., absolute pressure about 150-160 torr with a nitrogen sweep. The resulting 3.45 grams of Form A of 1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethanone obtained from the filter was confirmed by DSC.

Alternatively, Form A can be prepared by charging the amorphous S form to a vial containing about 18 mL of diisopropyl ether, 1.1 mL methanol, and Form A seeds. The reaction mixture was stirred. The reaction mixture was heated and cooled from 40° C. to 2° C., with cooling over about 4 hours and heating over about 1 hour, for a duration of six heating and cooling cycles. The reaction mixture was held at about 1° C. for 1 day. The mixture was reheated from 1° C. to 55° C. and then cooled to about 25° C. over a period of about 3.3 hours (approximately 200 minutes), and then cooled again to about 1° C. over a period of about 1-hour. The mixture was held at room temperature for about 24 hours then cooled to about 1° C. over about 30 minutes. The reaction mixture was held at 1° C. for several hours. The solids were transferred to a sintered glass fritted funnel and washed with cyclohexane. The solids were vacuum dried.

### HPLC Assay

Chiral HPLC of the sulfonamide enantiomers (90/10 (S/R)): Chiralpak IA column (250×3.0 mm), isocratic 50/50 methyl tert-butyl ether/ethanol with 0.2% diethylamine, flow rate 1.0 mL/minute, detection at 260 nm. Retention times: 8.5 minutes (S enantiomer) and 16.5 minutes (R enantiomer). The isolated solid is about 99% S and about 1% or less of the inactive isomer (R). Further enantiomeric enrichment can be obtained by stirring in MTBE (for example) and filtering any solids which form. Product was identical to the first eluting enantiomer of the racemate under the preparative chiral SFC conditions previously described. ¹H NMR, 600 MHz (d₆-DMSO): 7.88 (d, 2H), 7.82 (d, 1H), 7.73 (m, 2H), 5.18 (s, 2H), 4.62 (dd, 2H), 4.42 (dd, 2H), 4.28 (m, 4H), 3.20 (s, 3H); m/z(CI) 582 [M+H].

### Additional Chiral HPLC Assay Method

Chiracel AD-3R, 150×4.6 mm, 3 micron column. Flow rate of 1.5 mL per minute using a isocratic solvent mixture of 75:25 methanol:acetonitrile. Column temperature 40° C. Detection at 260 nm. Elution times are: S-isomer (4.0 minutes), R-isomer (7.8 minutes). Run time 15 minutes.

### HPLC Assay Method for the S-Isomer

ACE Excel 2 C18-AR, 150×4.6 mm column. Column temperature of 50° C. Detection at 260 nm. Flow rate is 1.5 mL per minute. Mobile phase A: 0.1% trifluoroacetic acid in water. Mobile phase B: 0.1% TFA in acetonitrile. Run at a gradient: initial time 45% B, 4.5 minutes 55% B, 20 minutes 100% B. Elution time of S-isomer is 9.8 minutes.

### BIOLOGICAL ASSAYS

A bioassay was performed on predominantly pre-adult and adult stage Lepeophtheirus salmonis (sea lice). Sea lice were exposed for 24 hours to the various concentrations of “test” compound (S-enantiomer, Example 1). Exposed (treated) and control lice were evaluated for response after 24 hours of exposure. Estimated EC₅₀ values were obtained, with 95% confidence intervals reported.

The results are shown in Table 1:

TABLE 1

| | | Adult |
|---|---|---|
| | Adult Males | Females |
| Compound | (EC₅₀) | (EC₅₀) |
| Test | 0.08 | 0.13 |
| Emamectin | 202 | 65 |
| (SLICE ®) | | |

Results: As shown in Table 1, the S-enantiomer of Example 1 displayed substantially greater activity against sea lice than emamectin (the active agent in SLICE®), with an EC₅₀ against male sea lice of 0.08 ppb and females, 0.13 ppb for generally chemical resistant sea lice. Conversely, emamectin showed substantially decreased potency against the same strain of sea lice, with an EC₅₀ of 202 ppb for adult males and 65 ppb for adult females. These results indicate that the S-enantiomer of Example 1 is significantly more potent against sea lice then the commercially available alternative (SLICE®).

1. A method for the treatment of a parasitic infection in a fish comprising administering to said fish an effective amount of a compound of Formula 1 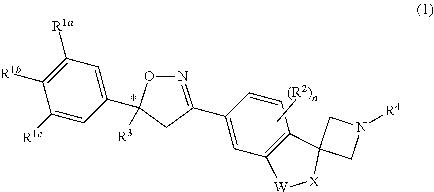
wherein
X and W are each independently O, S, NR⁶, —C(O)—, —C(NR⁷)—, or —C(S)—, when X is O, S, or NR⁶, then W is —CH₂—, —C(O)—, —C(NR⁷)—, or —C(S)—, and when W is O, S, or NR⁶, then X is —CH₂—, —C(O)—, —C(NR⁷)—, or —C(S)—;
R^{1a}, R^{1b}, and R^{1c} are each independently hydrogen, halo, hydroxyl, cyano, nitro, C₁-C₆alkyl, C₁-C₆haloalkyl, C₁-C₆alkoxy, C₀-C₃alkylC₃-C₆ cycloalkyl, C₁-C₆haloalkoxy, —C(O)NH₂, —SF₅, or —S(O)ₚR;
R² is halo, cyano, C₁-C₆alkyl, C₁-C₆haloalkyl, nitro, hydroxyl, —C(O)NR^{a}R^{b}, C₂-C₆alkenyl, C₂-C₆alkynyl, —S(O)R, or —OR;
R³ is cyano, C₁-C₆alkyl, C₁-C₆haloalkyl, —C(O)NR^{a}R^{b}, C₂-C₆alkenyl, C₂-C₆alkynyl, C₂-C₆haloalkenyl, or C₂-C₆haloalkynyl;
R⁴ is hydrogen, C₁-C₆alkyl, C₀-C₆alkylC₃-C₆cycloalkyl, —C(O)R⁵, —C(S)R⁵, —C(O)NR^{a}R⁵, —C(O)C(O)NR^{a}R⁵, —S(O)ₚR^{c}, —S(O)₂NR^{a}R⁵, —C(NR⁷)R⁵, —C(NR⁷)NR^{a}R⁵, C₀-C₆alkylphenyl, C₀-C₆alkylheteroaryl, or C₀-C₆alkylheterocycle;
R⁵ is hydrogen, C₁-C₆alkyl, C₂-C₆alkenyl, C₀-C₆alkylC₃-C₆cycloalkyl, C₀-C₆alkylphenyl, C₀-C₆alkylheteroaryl, or C₀-C₆alkylheterocycle;
R⁶ is hydrogen, C₁-C₆alkyl, hydroxyl, or C₁-C₆alkoxy;
R⁷ is hydrogen, C₁-C₆alkyl, hydroxyl, cyano, nitro, —S(O)ₚR^{c}, or C₁-C₆alkoxy;
R is C₁-C₆alkyl or C₃-C₆cycloalkyl optionally substituted with at least one halo substituent;
R^{a} is hydrogen, C₁-C₆alkyl, or C₀-C₃alkylC₃-C₆cycloalkyl; wherein the alkyl and alkylcycloalkyl is optionally substituted by cyano or at least one halo substituent;
R^{b} is hydrogen, C₁-C₆alkyl, C₃-C₆cycloalkyl, C₀-C₃alkylphenyl, C₀-C₃alkylheteroaryl, or C₀-C₃alkylheterocycle, each optionally substituted, where chemically possible, with at least one substituent selected from hydroxyl, cyano, halo, or —S(O)ₚR;
R^{c} is C₁-C₆alkyl, C₁-C₆haloalkyl, C₁-C₆haloalkylC₃-C₆cycloalkyl, C₀-C₃alkylC₃-C₆cycloalkyl, C₀-C₃alkylphenyl, C₀-C₃alkylheteroaryl, or C₀-C₃alkylheterocycle each optionally substituted with at least one substituent selected from cyano, halo, hydroxyl, oxo, C₁-C₆alkoxy, C₁-C₆haloalkoxy, C₁-C₆haloalkyl, —S(O)ₚR, —SH, —S(O)ₚNR^{a}R^{b}, —NR^{a}R^{b}, —NR^{a}C(O)R^{b}, —SC(O)R, —SCN, or —C(O)NR^{a}R^{b};
each of R⁴ and R⁵ C₁-C₆alkyl or C₀-C₆alkylC₃-C₆cycloalkyl moiety can be optionally and independently substituted by at least one substituent selected from cyano, halo, hydroxyl, oxo, C₁-C₆alkoxy, C₁-C₆haloalkoxy, C₁-C₆haloalkyl, C₁-C₆alkyl, hydroxylC₁-C₆alkyl-, —S(O)ₚR^{e}, —SH, —S(O)ₚNR^{a}R^{b}, —NR^{a}R^{b}, —NR^{a}C(O)R^{b}, —SC(O)R, —SCN, or —C(O)NR^{a}R^{b}; and
wherein each of R⁴ and R⁵ C₀-C₆alkylphenyl, C₀-C₆alkylheteroaryl, or C₀-C₆alkylheterocycle moiety can be further optionally substituted with at least one substituent selected from cyano, halo, oxo, ═S, ═NR⁷, hydroxyl, hydroxylC₁-C₆alkyl-, C₁-C₆alkoxy, C₁-C₆alkyl, C₁-C₆haloalkyl, —SH, —S(O)ₚR, and C₁-C₆haloalkoxy;
n is the integer 0, 1, or 2, and when n is 2, each R² may be identical or different from each other;
p is the integer 0, 1, or 2; and
wherein “*” is the chiral carbon, stereoisomers thereof, and veterinarily acceptable salts thereof.

2. The method of claim 1, wherein the compound is: 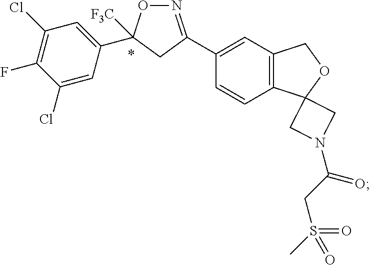 [SMILES: CS(=O)(=O)CC(=O)N1CC2(C1)OCc1cc(C3=NOC(c4cc(Cl)c(F)c(Cl)c4)(C(F)(F)F)C3)ccc12]
or a stereoisomer thereof, or a veterinarily acceptable salt thereof.

3. The method of claim 2, comprising the stereoisomer of the compound, wherein the stereoisomer is the S-enantiomer.

4. The method of claim 3, comprising the crystalline Form A of the S-enantiomer, the S-enantiomer, or the amorphous enantiomer prepared from crystalline Form A.

5. The method of claim 1, wherein the parasitic infection is from sea lice.

6. The method of claim 5, wherein the sea lice belongs to the genera Lepeophtheirus or Caligus and includes at least one of Lepeophtheirus salmonis, Caligus celmensi, Caligus curtus, Caligus dussumieri, Caligus elongates, Caligus longicaudatus, Caligus rogercresseyi or Caligus stromii.

7. The method of claim 1, wherein the fish is a farmed fish, wherein the fish is selected from the group consisting of carp, tuna, tilapia, cod, sole, bream, plaice, bass, halibut, catfish, trout, or salmon.

8. The method of claim 1, wherein the compound is administered to the fish orally through a feed composition.

9. The method of claim 8, wherein the feed composition is a pellet comprising fat, nutrients, protein and the compound.

10. The method of claim 1, wherein the compound is injected into the fish by intraperitoneal or intramuscular injection.

11. The method of claim 10, wherein the compound is co-administered with at least one of: an antigen, inactivated or killed virus or bacteria, or adjuvant.

12. The method of claim 11, wherein the compound is co-administered with several antigens in a polyvalent vaccine, optionally comprising one or more adjuvants.

13. The method of claim 1, wherein the compound is administered to the fish by immersing the fish in a solution comprising the compound.

14. The method of claim 13, wherein the compound is administered to the fish in a dose of at least 100 parts per billion (ppb).

15. The method of claim 1, wherein the compound is co-administered to the fish with an additional antiparasitic agent.

16. The method of claim 1, wherein the compound is administered to a plurality of fish.

17. A composition for oral administration to a fish comprising, the S-enantiomer of: 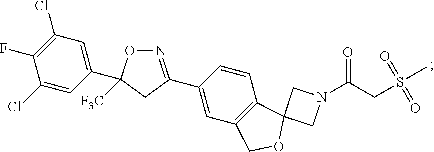 [SMILES: CS(=O)(=O)CC(=O)N1CC2(C1)OCc1cc(C3=NOC(c4cc(Cl)c(F)c(Cl)c4)(C(F)(F)F)C3)ccc12]
or a veterinarily acceptable salt thereof; and fish food.

18. The composition of claim 17, wherein the fish food comprises at least one of corn starch or oil.

19. The composition of claim 18, wherein the oil is herring oil or vegetable oil.

20. A kit comprising the composition of claim 17 and instructions for administration of the composition to fish and wherein the fish food comprises at least one of corn starch or oil.
